(19) United States
(12) Patent Application Publication
(10) Pub. No.: US 2016/0074153 A1
(43) Pub. Date: Mar. 17, 2016

(54) INTRAOCULAR LENS

(71) Applicant: ART, LIMITED, GRAND CAYMAN (KY)

(72) Inventors: Takayuki Akahoshi, Tokoyo (JP); Michael Southard, Sherwood, OR (US)

(73) Assignee: ART, LIMITED, GRAND CAYMAN (KY)

(21) Appl. No.: 14/843,118

(22) Filed: Sep. 2, 2015

Related U.S. Application Data
(60) Provisional application No. 62/050,450, filed on Sep. 15, 2014.

(51) Int. Cl. A61F 2/16

(52) U.S. Cl. CPC A61F 2/16; A61F 2002/1681

(57) ABSTRACT

A toric intraocular lens is configured for enhanced stability after implantation into a capsular bag of an eye. The intraocular lens has one or more haptics extending from the lens, with stabilization enhanced by configuring the lens to include at least one engagement element configured to cooperate with the interior to the capsular bag to resist relative rotation of the lens. In one aspect of the invention, the engagement element can be provided in the form of one or more rotation-inhibiting projections provided on the lens body and/or the haptics of the lens. In another aspect of the invention, the lens includes an anterior surface which is configured to provide enhanced frictional engagement of the intraocular lens with the interior of the eye. The anterior surface of the lens can be roughened in order to provide the desired frictional engagement.

### FIELD OF THE INVENTION

The present invention relates generally to the field of ophthalmic surgery, and more particularly, to an improved intraocular lens configured to resist rotation after implantation into the human eye.

### BACKGROUND OF THE INVENTION

When the eye becomes aged, diseased, or injured it may be necessary to remove the natural lens of the eye. Such removal is common for cataract surgery, in which a lens that has become clouded is removed. The removal of the natural lens of the eye may result in the loss or alteration of focused vision of a patient. Therefore, an artificial lens may be necessary to restore the vision of the patient. Some eyes have an oblong, irregularly shaped cornea that causes astigmatism, or blurred vision due to a refractive error in the eye.

Such artificial lenses may be provided in eyeglasses, contact lenses, or a permanent implant known as an intraocular lens (hereinafter “IOL”). The IOL is an artificial, generally circular lens with one or more stabilizing projections, arms, or haptics extending from the lens. A special type of implant called a toric IOL may be used to correct for astigmatism in the eye. The toric IOL has a lens body with an astigmatic axis that must be aligned with the steep corneal meridian of the eye. To implant the IOL in the eye, an incision is made in the anterior portion of the eye, typically while maintaining positive pressure within the eye to prevent collapse of the delicate structures of the eye. The IOL is generally folded or otherwise placed in a compressed state within an injector housing. The IOL may also be inserted into the eye in an unfolded state dependent upon the flexibility of the IOL material. In the case of an injector, the injector housing is elongate for being placed through the incision and into the patient's eye after the natural lens has been dismembered and aspirated, such as through phacoemulsification or laser generated surgery. A plunger is retained within the injector housing and is movable with respect to the housing. Movement of the plunger through the housing presses the IOL forward into the eye. The IOL, typically being resilient (with elastic memory), will subsequently expand to an uncompressed state upon entering the eye and exiting the injector. The haptics of the IOL serve to balance and center the IOL within the eye of the patient. The IOL is typically made from biocompatible materials such as PMMA, silicone, hydrogel, or acrylic.

During the removal of the natural lens of the eye, a physician may note that the zonules, or supporting ligaments of the capsular bag which contains the lens, are weakened, deteriorated, or otherwise insufficient to provide adequate structural support to centralize the haptics of the IOL within the eye. Therefore, a capsular tension apparatus or ring may be required to exert an outward pressure on the capsular bag prior to implantation of the IOL. Such capsular tension rings (CTR) are typically generally round or C-shaped and can be provided in various diameters to accommodate variations in human capsular bag diameters. Capsular tension rings are typically formed from a biocompatible implant material such as PMMA, silicone, hydrogels or acrylics. Other materials are also useful, depending upon the configuration of the rings, and how well they maintain their shape, and the size of the intended capsule. Capsular tension rings may be inserted through an incision in the anterior portion of the eye or may be folded and injected into the incision in a similar fashion as described above with respect to the IOL.

Today, the IOL designs are subject to rotation upon removal of the viscoelastic, as well as post-operatively when the capsule contracts and moves the IOL from its intended implant axis. In the former case, the eye is still surgically open, so additional manipulation of the lens can easily be performed to put it back on axis. However, if the lens moves due to improper sizing or capsular shrinkage (which occurs in virtually all eye surgery cases,) this also requires additional rotation. This is more complicated because the patient requires a re-operation to reposition the lens, and this subjects the patient to many surgical risks which include infections as well as intra-operative complications such as capsular tears, and other post-operative complications that follow such an event. The goal is to have a lens that stays where it is placed at the time of surgery, and that it can do so due to the lens design and the complementary CTR device.

To maintain the depth of the capsular bag during the implantation of the IOL, and thus maximize the ability of the surgeon to manipulate the anterior chamber throughout the implantation process, an ophthalmic viscoelastic device (hereinafter “OVD”) or material is used to fill the capsular bag. However, this OVD must be removed after the implantation of the IOL because it can lead to postoperative intraocular peaks in pressure. Removal of the OVD typically causes the IOL to shift or rotate in a clockwise direction due to the typical rotational asymmetry of the haptics. For the toric IOL, such shifting may result in degradation of the quality of the vision of the patient and require costly corrective surgical procedures that are inconvenient and may introduce additional risks to the patient.

### SUMMARY OF THE INVENTION

The inventor of the present invention has discovered an improved intraocular lens construction which is desirably configured to resist rotation of the lens after implantation into a human eye. This is achieved by providing the lens with one or more features which cooperate with the eye, or an associated capsular tension ring (CTR) to maintain the position of lens and abate its relative rotation, thereby enhancing the stabilization and centering of an implanted IOL within the eye. The present invention can not only find applicability for treatment of astigmatism, but also for treatment for eyes where the capsular forces of the bag have been compromised due to an irregular rhexis, an irregular capsule, or zonular dehiscence or tearing, or situations in which the capsule may not provide 360 degree support of the implanted IOL.

In accordance with the present invention, an intraocular lens comprises a generally circular, central lens body having first and second, opposite anterior and posterior surfaces. At least one of these surfaces has an arcuate cross-sectional configuration for providing vision correction for a patient.

The present lens further includes at least one haptic, and may comprise a pair of haptics, extending from the central lens body generally at diametrically opposed portions thereof. Each of the haptics has an arcuate, elongated configuration so that an outwardly facing surface of each haptic is engageable with the interior of the eye, or an associated CTR, for stabilizing the lens and limiting relative rotation thereof. More than two haptics, or a totally-encircling haptic (e.g. a skirt), as well as other configurations, can be employed.

In accordance with the present invention, the intraocular lens includes at least one engagement element for engagement with the interior of the eye to limit rotation of the intraocular lens.

In accordance with one aspect of the present invention, each of the haptics of the lens has at least one engagement element in the form of a rotation-inhibiting projection extending from the outwardly facing surface thereof for engagement with the interior of the eye to limit rotation of the intraocular lens. In accordance with the illustrated embodiment, each of the rotation-inhibiting projections comprises a gear-like protuberance or tooth, or knob-like protuberance extending from the respective one of the haptics. Preferably, each of the protuberances extends in a direction generally opposite to a direction in which the respective one of said haptics extends from said central lens body. Each haptic may be provided with a plurality of the rotation-inhibiting projections. The protuberances are desirably configured to confirm meshing with a complementary CTR device, that provides the desired anti-rotation characteristics.

In accordance with another aspect of the present invention, the posterior surface of the central lens body of the lens has an annular surface which is configured to provide the engagement element of the intraocular lens. In one form, the engagement element comprises an annular surface of the posterior surface of the lens body which is configured to enhance frictional engagement of the intraocular lens with the interior of the eye, to further limit rotation of the intraocular lens. In a current embodiment, the posterior surface of the central lens body has annular surface which is roughened to enhance frictional engagement with the interior of the eye.

In an alternate embodiment, the annular surface of the anterior surface of the lens body includes an engagement element in the form of at least one forwardly-extending projection for limiting rotation of the intraocular lens. In the preferred form, the forwardly-extending projection is configured for engagement with an opening in the capsular bag that has been created by a femtosecond laser. In the illustrated embodiment, a pair of forwardly-extending projections are provided in diametrically opposed relationship for respective engagement with a pair of openings created by the femtosecond laser along the axis of astigmatism.

In another illustrated embodiment, the peripheral portion of the central lens body of the lens includes the engagement element in the form of at least one outwardly-extending, rotation-inhibiting projection extending from the peripheral portion, with the illustrated form including a pair of the rotation-inhibiting projections arranged on the peripheral portion of the central lens body in diametrically opposed relationship.

Other features and advantages of the present invention will become readily apparent from the following detailed description, the accompanying drawings, and the appended claims. Better understood with reference to the accompanying figures and detailed description.

### BRIEF DESCRIPTION OF THE DRAWINGS

FIG. 1 is a cross-sectional diagrammatic view of a human eye

FIG. 2 is a top plan view of a toric, intraocular lens embodying the principles of the present invention;

FIG. 3 is a relatively enlarged, fragmentary perspective view of the intraocular lens shown in FIG. 2;

FIG. 4 is a rear, plan view of an alternate embodiment of the present intraocular lens;

FIG. 5 is a side elevation view of the intraocular lens shown in FIG. 4;

FIG. 6 is a top plan view of an alternate embodiment of a toric, intraocular lens embodying the principles of the present invention;

FIG. 7 is a relatively enlarged, fragmentary perspective view of the intraocular lens shown in FIG. 6;

FIG. 8 is a top plan view of a further embodiment of a toric, intraocular lens embodying the principles of the present invention;

FIG. 9 is a relatively enlarged, fragmentary perspective view of the intraocular lens shown in FIG. 8;

FIG. 10 is a top plan view of a further embodiment of a toric, intraocular lens embodying the principles of the present invention;

FIG. 11 is a relatively enlarged, fragmentary perspective view of the intraocular lens shown in FIG. 10; and

FIG. 12 is a diagrammatic view illustrating a pair of openings formed by a femtosecond laser in the anterior capsule of an eye for respectively receiving rotation-inhibiting projections of the embodiment of the toric intraocular lens shown in FIG. 10.

### DETAILED DESCRIPTION OF THE PREFERRED EMBODIMENTS

While the present invention is susceptible of embodiment in various forms, there is shown in the drawings and will hereinafter be described presently preferred embodiments of the invention, with the understanding that the present disclosure is to be considered an exemplification of the invention, and is not intended to limit the invention to the specific embodiments illustrated.

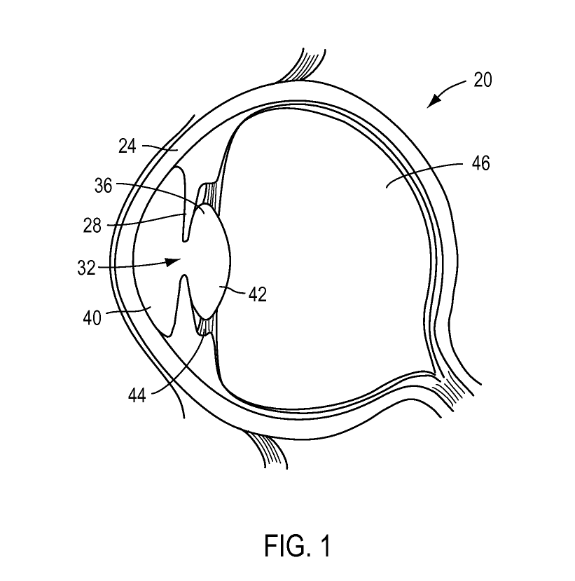

FIG. 1 shows a diagrammatic cross-sectional view of the human eye 20. Beginning at the exterior of the eye 20, the eye 20 has a protective outer layer or cornea 24 which retains the fluids or aqueous humor of the eye 20. Inward of the cornea 24 is the ring-like iris 28 with an aperture or pupil 32 for restricting light reaching the lens 36. The lens 36 is located within a front or anterior cavity 40 of the eye 20 and is encased in a capsular bag 42. Supporting ligaments or zonules 44 stabilize and center the capsular bag 42 within the eye 20. Opposing the anterior cavity 40 of the eye 20 is the posterior cavity 46 containing the optical nerves and arteries of the eye 20. When the lens 36 has been removed from the eye 20, such as through phacoemulsification, some or all of the capsular bag 42 remains connected to the zonules 44 in the eye 20.

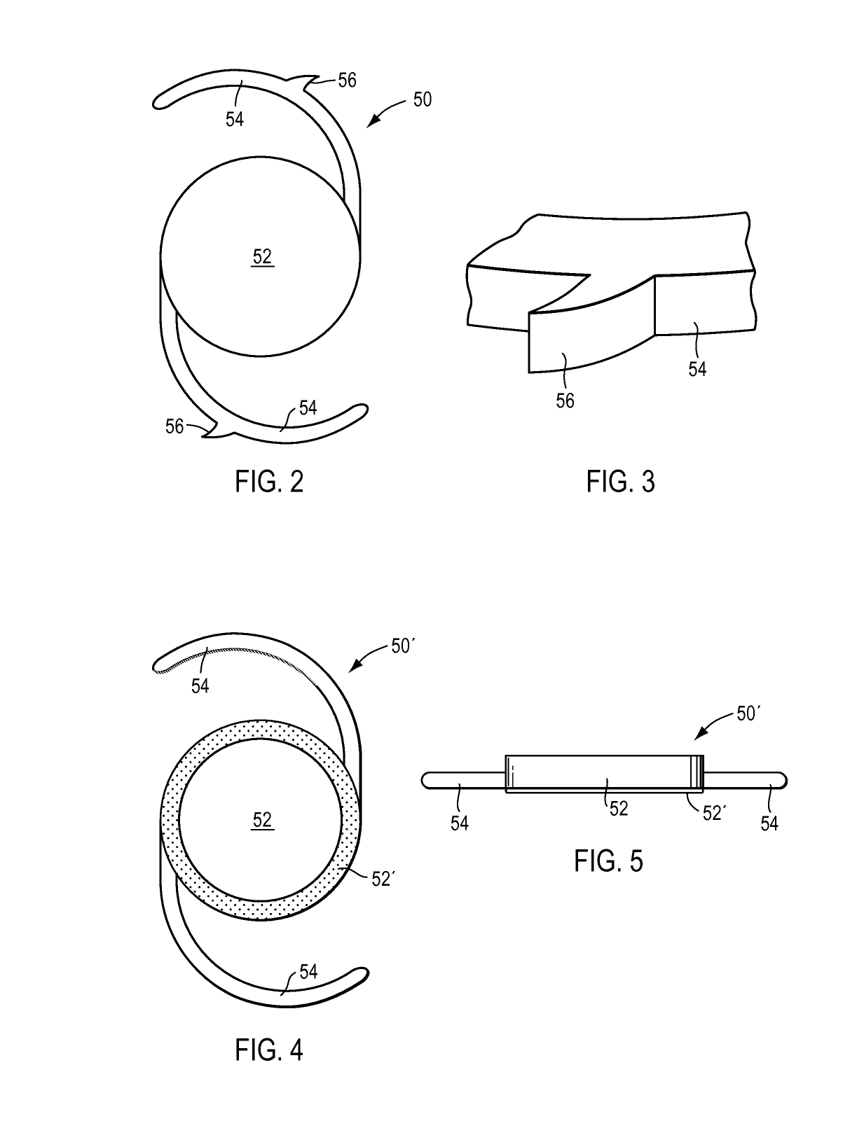

FIG. 2 shows a toric intraocular lens 50 embodying the principles of the present invention. Attendant to a phacoemulsification procedure for removal to the natural lens, intraocular lens (IOL) 50 is implanted in the capsular bag 42 of the patient. The lens 50 is made from a biocompatible material such as PMMA, silicone, hydrogel, or acrylic, and has a central lens body 52 having first and second, opposite posterior (posterior with respect to the frontal plane) and anterior (anterior with respect to the frontal plane) surfaces. At least one of these surfaces has an accurate cross-sectional configuration for providing vision correction for the patient.

The lens body 52 may have one or more surfaces of a varying degree of convexity depending on the need for correction to the patient's vision. The toric IOL has an astigmatic axis that must be aligned with the steep corneal meridian.

The inventor of the present invention has discovered an improved intraocular lens construction which is desirably configured to resist rotation of the lens after implantation into a human eye, specifically when the OVD is removed from the capsular bag. Resisting rotation is achieved by providing the lens with one or more features which cooperate with the eye to maintain the position of lens and abate its relative rotation, thereby enhancing the stabilization and centering of an implanted IOL within the eye.

In accordance with this aspect of the present invention, the lens 50 includes a pair of haptics 54 extending from the central lens body 52 generally at diametrically opposed portions thereof. Each of the haptics 54 has an arcuate, elongated configuration so that an outwardly facing surface of each haptic is engageable with the interior of the eye for stabilizing the lens 50 and limiting relative rotation thereof. The two generally arcuate haptics 54 extend generally radially outward from the exterior surface of the lens body 52. The haptics 54 are intended to contact the capsular bag to center the lens body 52 within the eye 20, or may be configured to engage and cooperate with an associated capsular tension ring (CTR).

In accordance with this embodiment of the present invention, intraocular lens 50 includes at least one engagement element which is engageable with the interior of the eye or an associated CTR for limiting rotation of the lens. In the embodiment illustrated in FIGS. 2 and 3, each of the haptics 54 of the lens 50 has an engagement element comprising at least one rotation-inhibiting projection extending from the outwardly facing surface thereof for engagement with the interior of the eye to limit rotation of the intraocular lens. In accordance with the illustrated embodiment, each of the rotation-inhibiting projections comprises a protuberance 56 extending from the respective one of the haptics 54. Preferably, each of the protuberances 56 extends in a direction generally opposite to a direction in which the respective one of said haptics 54 extends from said central lens body 52. Each protuberance can be configured to provide the desired engagement and cooperation with the eye or a CTR, and to this end, may have a generally attenuated, tooth-like configuration, and may configured generally in the nature of a gear tooth.

In the embodiment illustrated in FIGS. 8 and 9, the intraocular lens 50 includes a pair of haptics 54 each including a plurality of the rotation-inhibiting projections or protuberances, designated 56′, extending in spaced apart relationship along the outwardly facing surface of the respective one of the haptics 54. This arrangement can act to further inhibit rotation of the lens 50.

In accordance with another aspect of the present invention, shown in FIGS. 4 and 5, an intraocular lens 50′ includes a central lens body 52 having an anterior surface. In this embodiment, the anterior surface of the lens body provides the engagement element in the form of an annular surface which is configured to provide enhance frictional engagement of the intraocular lens with the interior of the eye, to further limit rotation of the intraocular lens. In a current embodiment, the anterior surface of the central lens body has annular surface 52′ which is roughened to enhance frictional engagement with the interior of the eye. The annular surface 52′ could alternatively be formed from a higher tack material than the lens body 50 so as to provide an improved adhesion to the capsular surface. It will be appreciated that the annular surface 52′ need not be fully annular, could be comprised of a plurality of discrete surfaces, and could be optimally located on the lens body 50 so as to engage the capsular surface.

For the toric IOL 50, 50′, a one degree of misalignment of the toric IOL with the astigmatic axis of the steep corneal meridian of the eye may result in a loss of over 3% of the toric effect (for correction of the astigmatism of the eye). Furthermore, rotational misalignment of the toric IOL astigmatic axis with the steep corneal meridian of the eye of over 30% may result in a total loss of toric effect, and may even induce astigmatism. The inventor has discovered that stabilization of the IOL, and inhibiting relative rotation of the lens within the capsular bag, can inhibit undesirable operative and/or post-operative movement of the toric IOL.

In the broadest concept of the invention, the projections provided in the form of protuberances 56 may take a variety of geometries such as straight or angled cantilevered beams projecting from any surface of the haptics 54. Such beams may have shapes that are fully arcuate, partially arcuate, or polygonal. Furthermore, the rotation-inhibiting projections may be comprised of hooks or loops for engaging the interior of the capsular bag. The projections need not be unitarily formed with the lens body, but instead may be attached to the body in a secondary process, or made from a different biocompatible material. Furthermore, the projections may engage one or more cooperating projections of an implanted CTR (not illustrated).

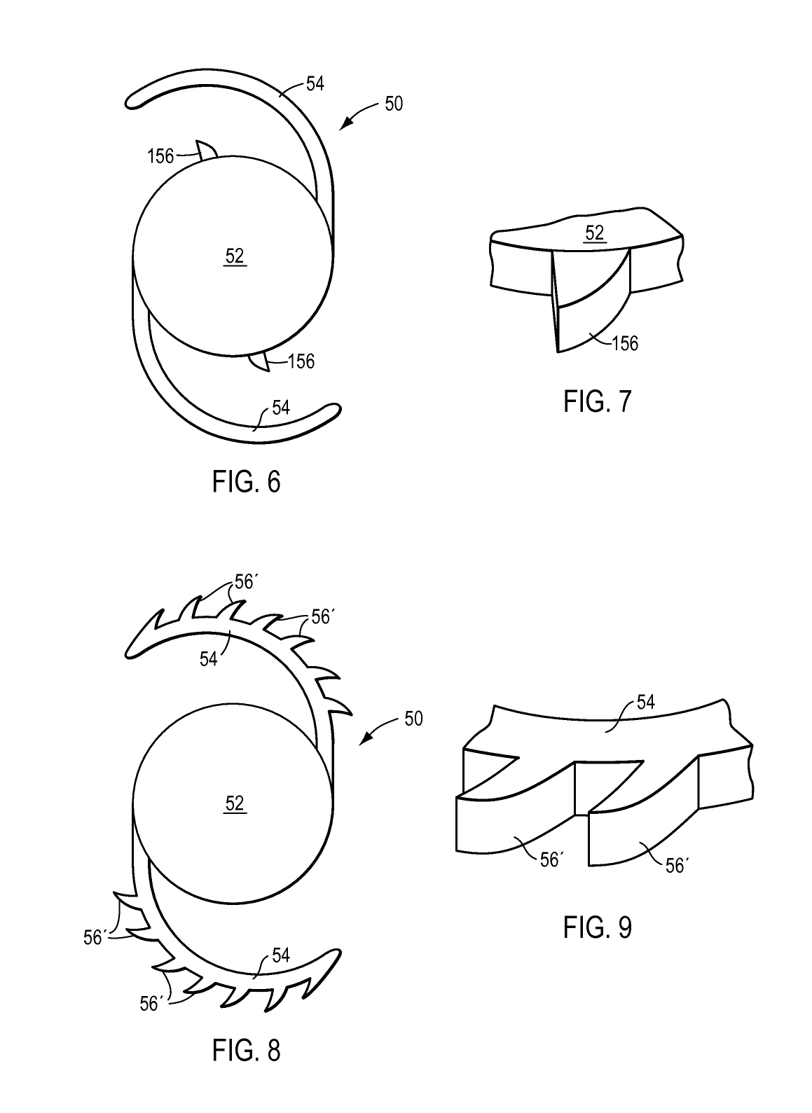

In another embodiment of the present invention shown in FIGS. 6 and 7, the peripheral portion of the central lens body 52 of the lens 50 includes the engagement element in the form of at least one outwardly-extending, rotation-inhibiting projection 156 extending from the peripheral portion, with the illustrated form including a pair of the rotation-inhibiting projections 156 arranged on the peripheral portion of the central lens body in diametrically opposed relationship. Like the previously described projections 56, 56′, projections 156 may be provided with an attenuated configuration and be configured as a tooth.

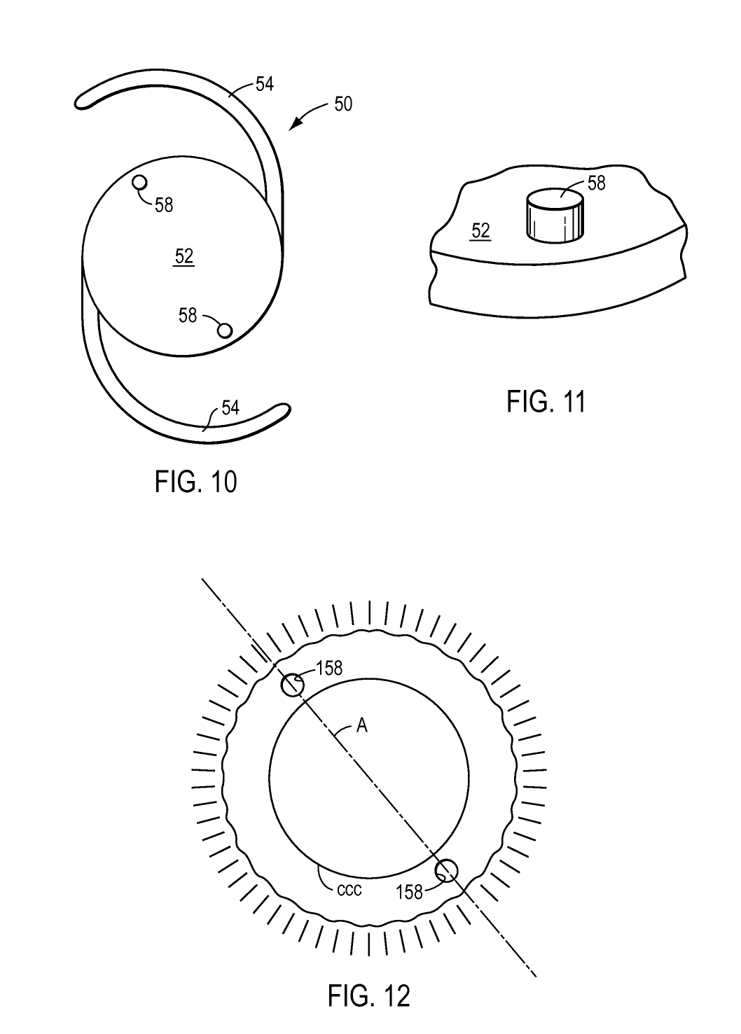

In a further alternate embodiment of the present invention, shown in FIGS. 10-12, the annular portion of the outermost or anterior surface of the lens body 52 is configured to include an engagement element in the form of at least one forwardly-extending projection 58 for limiting rotation of the intraocular lens 50. In the preferred form, the forwardly-extending projection 58 is configured for engagement with an opening 158 created by a femtosecond laser in anterior capsule or capsular bag of the eye, in the region of the femtosecond laser continuous curvilinear capsulorrhexis (“CCC” in FIG. 12). In this illustrated embodiment, a pair of the forwardly-extending projections 58 are provided in diametrically opposed relationship for respective engagement a pair of openings 158 created by the femtosecond laser along the axis “A” of an astigmatism.

The method of implantation and operation of the inventive lens 50, 50′ will now be discussed. After the capsular bag 42 has been incised and nucleus or lens 36 of the eye has been removed, the OVD is implanted into the capsular bag and a ring-like capsular tensioning device can optionally be implanted. The physician performing the lens implantation will insert the foldable lens 50, 50′ into the capsular bag in a compressed or folded condition. If the lens 50, 50′ is made from PMMA, then it cannot be folded during implantation. After insertion, the lens generally assumes the illustrated orientation, with the haptics 54 extending from the body of the lens for engagement with the interior of the capsular bag for stabilizing the lens. The IOL is aligned by the physician. During removal of the OVD, relative rotation of the lens (away from the aligned position) is desirably inhibited by the provision of the engagement elements such as the: (1) projections 56, 56′, and 156, and/or (2) the friction-enhancing surface In accordance with the present invention, and/or (3) the forwardly-extending projections 58.

From the foregoing, it will be observed that numerous modifications and variations can be effected with departing from the true spirit and scope of the novel concept of the present invention. It is to be understood that the present disclosure is to be considered an exemplification of the invention, and is not intended to limit the invention to the specific embodiments illustrated. The disclosure is intended to cover by the appended claims all such modifications as fall with the scope of the claims.

1. An intraocular lens, comprising:
a generally circular, central lens body having first and second, opposite posterior and anterior surfaces, at least one of said surfaces having an arcuate cross-sectional configuration for providing vision correction for a patient; and
at least one haptic extending from said central lens body said haptic having an arcuate, configuration so that an outwardly facing surface of each haptic is engageable with the interior the of the eye or an associated capsular tension ring for stabilizing said lens and limiting rotation thereof, said intraocular lens including at least one engagement element to limit rotation of said intraocular lens.

2. An intraocular lens in accordance with claim 1, wherein
said lens includes a pair of said haptics extending from said central lens body generally at diametrically opposed portions thereof, each of said haptics having an arcuate, elongate configuration, each of said haptics at least one of said engagement elements to limit rotation of said intraocular lens.

3. An intraocular lens in accordance with claim 1, wherein
said posterior surface of said central lens body has an annular surface which is configured to provide said engagement element to enhance frictional engagement of said intraocular lens with the interior of the eye to further limit rotation of said intraocular lens.

4. An intraocular lens in accordance with claim 3, wherein
said annular surface of said posterior surface of said central lens body is roughened to provide said engagement element to enhance frictional engagement with the interior of the eye.

5. An intraocular lens in accordance with claim 1, wherein
said at least one engagement element is at least one forwardly-extending projection extending from said anterior surface of said central lens body for limiting rotation of said intraocular lens.

6. An intraocular lens in accordance with claim 5, wherein
said at least one forwardly-extending projection is configured for engagement with an opening in the eye created by a femtosecond laser.

7. An intraocular lens in accordance with claim 6, wherein
said anterior surface of said central lens body includes a pair of said forwardly-extending projections arranged in a diametrically opposed relationship, said pair of forwardly-extending projections being configured for respective engagement in a pair of said openings created by a femtosecond laser on an axis of an astigmatism of the eye.

8. An intraocular lens in accordance with claim 1, wherein
said at least one engagement element extends from said at least one haptic in the form of a rotation-inhibiting projection extending from said outwardly facing surface of said at least one haptic.

9. An intraocular lens in accordance with claim 8, wherein
each of said haptics includes a plurality of said rotation-inhibiting projections extending from said outwardly facing surface thereof.

10. An intraocular lens in accordance with claim 8, wherein
said at least one rotation-inhibiting projection comprises a tooth extending from the respective one of said haptics.

11. An intraocular lens in accordance with claim 10, wherein
said tooth extends from said haptic at an angle for engaging the eye to oppose rotation of said central lens body.

12. An intraocular lens in accordance with claim 11, wherein
said tooth extends from said haptic at an interior angle between 45 and 90 degrees.

13. An intraocular lens in accordance with claim 1, wherein
said engagement element comprises a rotation-inhibiting projection extending outwardly from a peripheral portion of said central lens body.

14. An intraocular lens in accordance with claim 13, wherein
said peripheral portion of said central lens body includes a pair of diametrically opposed rotation-inhibiting projections.
